SEQUENCE LISTING (9 sequences)

SEQ ID NO: 1 - protein, 374 aa, Artificial Sequence
  truncated derivative of a full-length S. aureus
TNSDVTPVQAANQYGYAGLSAAYEPTSAVNVSQTGQLLYQYNIDTKWNPASMTKLMTMYLTLEAVNKGQLSLDDTVTMTNKEYIMSTLPELSNTKLYPGQVWTIADLLQITVSNSSNAAALILAKKVSKNTSDFVDLMNNKAKAIGMKNTHFVNPTGAENSRLRTFAPTKYKDQERTVTTARDYAILDLHVIKETPKILDFTKQLAPTTHAVTYYTFNFSLEGAKMSLPGTDGLKTGSSDTANYNHTITTKRGKFRINQVIMGAGDYKNLGGEKQRNMMGNALMERSFDQYKYVKILSKGEQRINGKKYYVENDLYDVLPSDFSKKDYKLVVEDGKVHADYPREFINKDYGPPTVEVHQPIIQKANTVAKSMWE

SEQ ID NO: 2 - protein, 385 aa, Artificial Sequence
  derivative of SEQ ID NO 1 containing an amino terminus methinonine-alanine-serine addition and carboxyl His-Tag
MASTNSDVTPVQAANQYGYAGLSAAYEPTSAVNVSQTGQLLYQYNIDTKWNPASMTKLMTMYLTLEAVNKGQLSLDDTVTMTNKEYIMSTLPELSNTKLYPGQVWTIADLLQITVSNSSNAAALILAKKVSKNTSDFVDLMNNKAKAIGMKNTHFVNPTGAENSRLRTFAPTKYKDQERTVTTARDYAILDLHVIKETPKILDFTKQLAPTTHAVTYYTFNFSLEGAKMSLPGTDGLKTGSSDTANYNHTITTKRGKFRINQVIMGAGDYKNLGGEKQRNMMGNALMERSFDQYKYVKILSKGEQRINGKKYYVENDLYDVLPSDFSKKDYKLVVEDGKVHADYPREFINKDYGPPTVEVHQPIIQKANTVAKSMWELEHHHHHH

SEQ ID NO: 3 - protein, 431 aa, S. aureus
MKNLISIIIILCLTLSIMTPYAQATNSDVTPVQAANQYGYAGLSAAYEPTSAVNVSQTGQLLYQYNIDTKWNPASMTKLMTMYLTLEAVNKGQLSLDDTVTMTNKEYIMSTLPELSNTKLYPGQVWTIADLLQITVSNSSNAAALILAKKVSKNTSDFVDLMNNKAKAIGMKNTHFVNPTGAENSRLRTFAPTKYKDQERTVTTARDYAILDLHVIKETPKILDFTKQLAPTTHAVTYYTFNFSLEGAKMSLPGTDGLKTGSSDTANYNHTITTKRGKFRINQVIMGAGDYKNLGGEKQRNMMGNALMERSFDQYKYVKILSKGEQRINGKKYYVENDLYDVLPSDFSKKDYKLVVEDGKVHADYPREFINKDYGPPTVEVHQPIIQKANTVAKSMWEEHPLFTIIGGTCLVAGLALIVHMIINRLFRKRK

SEQ ID NO: 4 - protein, 431 aa, S. aureus
MKNLISIIIILCLTLSIMTPYAQAANSDVTPVQAANQYGYAGLSAAYEPTSAVNVSQTGQLLYQYNIDTKWNPASMTKLMTMYLTLEAVNKGQLSLDDTVTMTNKEYIMSTLPELSNTKLYPGQVWTIADLLQITVSNSSNAAALILAKKVSKNTSDFVDLMNNKAKAIGMKNTHFVNPTGAENSRLRTFAPTKYKDQERTVTTARDYAILDLHVIKETPKILDFTKQLAPTTHAVTYYTFNFSLEGAKMSLPGTDGLKTGSSDTANYNHTITTKRGKFRINQVIMGAGDYKNLGGEKQRNMMGNALMERSFDQYKYVKILPKGEQRINGKKYYVENDLYDVLPSDFSKKDYKLVVEDGKVHADYPREFINKDYGPPTVEVHQPIIQKANTVAKSMWEEHPLFTIIGGACLVAGLALIVHMIINRLFRKRK

SEQ ID NO: 5 - protein, 431 aa, S. aureus
MKNLISIIIILCLTLSIMTPYAQATNSDVTPVQAANQYGYAGLSAAYEPTSAVNVSQTGQLLYQYNIDTKWNPASMTKLMTMYLTLEAVNKGQLSLDDTVTMTNKEYIMSTLPELSNTKLYPGQVWTIADLLQITVSNSSNAAALILAKKVSKNTSDFVDLMNNKAKAIGMKNTHFVNPTGAENSRLRSFAPTKYKDQERTVTTARDYAILDLHVIKETPKILDFTKQLAPTTHAVTYYTFNFSLEGAKMSLPGTDGLKTGSSDTANYNHTITTKRGKFRINQVIMGAGDYKNLGGEKQRNMMGNALMERSFDQYKYVKILSKGEQRINGKKYYVENDLYDVLPSDFSKKDYKLVVEDGKVHADYPREFINKDYGPPTVEVHQPIIQKANTVAKSMWEEHPLFTIIGGACLVAGLALIVHMIINRLFRKRK

SEQ ID NO: 6 - protein, 431 aa, S. aureus
MKNLISIIIILCLTLSIMTPYAQAANSDVTPVQAANQYGYAGLSAAYEPTSAVNVSQTGQLLYQYNIDTKWNPASMTKLMTMYLTLEAVNKGQLSLDDTVTMTNKEYIMSTLPELSNTKLYPGQVWTIADLLQITVSNSSNAAALILAKKVSKNTSDFVDLMNNKAKAIGMKNTHFVNPTGAENSRLRSFAPTKYKDQERTVTTARDYAILDLHVIKETPKILDFTKQLAPTTHAVTYYTFNFSLEGAKMSLPGTDGLKTGSSDTANYNHTITTKRGKFRINQVIMGAGDYKNLGGEKQRNMMGNALMERSFDQYKYVKILSKGEQRINGKKYYVENDLYDVLPSDFSKKDYKLVVEDGKVHADYPREFINKDYGPPTVEVHQPIIQKANTVAKSMWEEHPLFTIIGGACLVAGLALIVHMIINRLFRKRK

SEQ ID NO: 7 - protein, 431 aa, S. aureus
MKNLISIIIILCLTLSIMTPYAQATNSDVTPVQAANQYGYAGLSAAYEPTSAVNVSQTGQLLYQYNIDTKWNPASMTKLMTMYLTLEAVNKGQLSLDDTVTMTNKEYIMSTLPELSNTKLYPGQVWTIADLLQITVSNSSNAAALILAKKVSKNTSDFVDLMNNKAKAIGMKNTHFVNPTGAENSRLRTFAPTKYKDQEATVSTARDYAILDLHVIKETPKILDFTKQLAPTTHAVTYYTFNFSLEGAKMSLPGTDGLKTGSSDTANYNHTITTKRGKFRINQVIMGAGDYKNLGGEKQRNMMGNALMERSFDQYKYVKILSKGEQRINGKKYYVENDLYDVLPSDFSKKDYKLVVEDGKVHADYPREFINKDYGPPTVEVHQPIIQKANTVAKSMWEEHPLFTIIGGTCLVAGLALIVHMIINRLFRKRK

SEQ ID NO: 8 - protein, 431 aa, S. aureus
MKNLISIIIILCLTLSIMTPYAQATNSDVTPVQAANQYGYAGLSAAYEPTSAVNVSQTGQLLYQYNIDTKWNPASMTKLMTMYLTLEAVNKGQLSLDDTVTMTNKEYIMSTLPELSNTKLYPGQVWTIADLLQITVSNSSNAAALILAKKVSKNTSDFVDLMNNKAKAIGMKNTHFVNPTGAENSRLRTFAPTKYKDQERTVTTARDYAILDLHVIKETPKILDFTKQLAPTTLAVTYYTFNFSLEGAKMSLPGTDGLKTGSSDTANYNHTITTKRGKFRINQVIMGAGDYKNLGGEKQRNMMGNALMERSFDQYKYVKILSKGEQRINGKKYYVENDLYDVLPSDFSKKDYKLVVEDGKVHADYPREFINKDYGPPTVEVHQPIIQKANTVAKSMWEEHPLFTIIGGTCLVAGLALIVHMIINRLFRKRK

SEQ ID NO: 9 - DNA, 1158 bp, Artificial Sequence
  nucleic acid encoding SEQ ID NO 2
atggctagcactaacagtgacgtaacccctgtacaagcagcaaatcaatatggttatgcaggtttgtcggctgcatacgaaccgacgagtgctgttaatgtaagtcaaactggacaattactgtatcaatacaatatcgatactaagtggaatccagcgtctatgactaaattaatgacaatgtacttaacattggaagctgtaaataaggggcagctttcacttgatgacacagtcacaatgacgaacaaagaatatattatgtctacactacctgagttgagtaatacgaaactatatcctggacaagtatggacaatcgcagacctattacaaattacagtatctaattctagtaatgccgcggcattaattttagctaagaaggtttcaaaaaacaccagcgatttcgttgatttaatgaataacaaagctaaagctatcggaatgaaaaatacacatttcgtcaatccaacgggtgctgaaaattcaagattacgtacatttgcaccaacaaagtataaagaccaagaacgtactgtaacgactgctagagactatgccattttagatttacacgtgattaaagagacacctaaaatattagactttacaaagcagttagcaccaacaacgcatgcagttacgtattacacattcaacttttcattggaaggtgcaaaaatgagtttgccgggtacagatggtttaaaaactggatcaagtgatacagcaaattacaaccatacgattactaccaaacgaggtaaatttagaattaatcaagttatcatgggtgcaggagactataaaaaccttggtggcgagaagcaacgtaatatgatggggaatgcattaatggaacgctcatttgatcagtataaatatgtaaaaatattgtctaaaggtgagcaaaggataaatggtaagaaatattatgttgaaaatgatctttacgatgttttaccaagtgattttagtaaaaaagattataaacttgtagtcgaagatggtaaagtacacgcggactatccaagagaatttattaataaagattatggacctccaactgtagaagttcatcagccaattattcaaaaggcaaatactgttgctaaaagtatgtgggaactcgagcaccaccaccaccaccactga

(19) United States
(12) Patent Application Publication
(10) Pub. No.: US 2007/0264278 A1
(43) Pub. Date: Nov. 15, 2007

(54) Polypeptides for Inducing a Protective Immune Response Against Staphylococcus Aureus

(76) Inventors: Annaliesa S. Anderson, Doylestown, PA (US); Donna L. Montgomery, Chalfont, PA (US)

Correspondence Address: MERCK AND CO., INC, P O BOX 2000, RAHWAY, NJ, 07065-0907, US

(21) Appl. No.: 11/663,037

(22) PCT Filed: Sep. 13, 2005
(86) PCT No.: PCT/US05/32744

§ 371 Date: Mar. 15, 2007

Related U.S. Application Data
(60) Provisional application No. 60/610,814, filed on Sep. 17, 2004.

(51) Int. Cl. A61K 39/395; A61P 37/00; C07H 21/00; C07K 14/00; C12N 1/00; C12N 5/00; C12P 21/00

(52) U.S. Cl. 424/190.1; 435/243; 435/325; 435/71.2; 530/324; 536/23.1

(57) ABSTRACT

The present invention features polypeptides comprising an amino acid sequence structurally related to SEQ ID NO: 1 and uses of such polypeptides. SEQ ID NO: 1 is a truncated derivative of a full-length S. aureus polypeptide. The full-length polypeptide is referred to herein as full-length penicillin-binding protein 4 (“PBP4”). A His-tagged derivative of SEQ ID NO: 1 was found to produce a protective immune response against S. aureus.

### CROSS-REFERENCE TO RELATED APPLICATIONS

The present application claims the benefit of U.S. Provisional Application No. 60/610,814 filed Sep. 17, 2004, which is hereby incorporated by reference herein.

### BACKGROUND OF THE INVENTION

The references cited throughout the present application are not admitted to be prior art to the claimed invention.

Staphylococcus aureus is a pathogen responsible for a wide range of diseases and conditions. Examples of diseases and conditions caused by S. aureus include bacteremia, infective endocarditis, folliculitis, furuncle, carbuncle, impetigo, bullous impetigo, cellulitis, botryomyosis, toxic shock syndrome, scalded skin syndrome, central nervous system infections, infective and inflammatory eye disease, osteomyletitis and other infections of joints and bones, and respiratory tract infections. (The Staphylococci in Human Disease, Crossley and Archer (eds.), Churchill Livingstone Inc. 1997.)

Immunological based strategies can be employed to try to control S. aureus infections and the spread of S. aureus. Immunological based strategies include passive and active immunization. Passive immunization employs transfer of immunoglobulins targeting S. aureus. Active immunization induces immune responses against S. aureus.

Potential S. aureus vaccines target S. aureus polysaccharides and polypeptides. Targeting can be achieved using suitable S. aureus polysaccharides or polypeptides as vaccine components. Examples of potential polysaccharides vaccine components include S. aureus type 5 and type 8 capsular polysaccharides. (Shinefield et al., N. Eng. J. Med. 346:491-496, 2002.) Examples of polypeptides that may be employed as possible vaccine components include collagen adhesin, fibrinogen binding proteins, and clumping factor. (Mamo et al., FEMS Immunology and Medical Microbiology 10:47-54, 1994, Nilsson et al., J. Clin. Invest. 101:2640-2649, 1998, Josefsson et al., The Journal of Infectious Diseases 184:1572-1580, 2001.)

Information concerning S. aureus polypeptide sequences has been obtained from sequencing the S. aureus genome. (Kuroda et al., Lancet 357:1225-1240, 2001, Baba et al., Lancet 359:1819-1827, 2000, Kunsch et al., European Patent Publication EP 0 786 519, published Jul. 30, 1997.) To some extent bioinformatics has been employed in efforts to characterize polypeptide sequences obtained from genome sequencing. (Kunsch et al., European Patent Publication EP 0 786 519, published Jul. 30, 1997.)

Techniques such as those involving display technology and sera from infected patients have been used as part of an effort to try to identify genes coding for potential antigens. (Foster et al., International Publication Number WO 01/98499, published Dec. 27, 2001, Meinke et al., International Publication Number WO 02/059148, published Aug. 1, 2002, Etz et al., PNAS 99:6573-6578, 2002.)

### SUMMARY OF THE INVENTION

The present invention features polypeptides comprising an amino acid sequence structurally related to SEQ ID NO: 1 and uses of such polypeptides. SEQ ID NO: 1 is a truncated derivative of a full-length S. aureus polypeptide. The full-length polypeptide is referred to herein as full-length penicillin-binding protein 4 (“PBP4”). A His-tagged derivative of SEQ ID NO: 1 was found to produce a protective immune response against S. aureus.

Reference to “protective” immunity or immune response indicates a detectable level of protection against S. aureus infection. The level of protection can be assessed using animal models such as those described herein.

Thus, a first aspect of the present invention describes a polypeptide immunogen comprising an amino acid sequence at least 85% identical to SEQ ID NO: 1, wherein the polypeptide does not contain a carboxyl terminus provided by amino acids 398-431 of SEQ ID NO: 3 and the polypeptide provides protective immunity against S. aureus. SEQ ID NO: 3 provides a full length PBP4 polypeptide, wherein amino acids 398-431 provide a carboxyl terminus domain.

Reference to “immunogen” indicates the ability to provide protective immunity.

Reference to comprising an amino acid sequence at least 85% identical to SEQ ID NO: 1 wherein the polypeptide does not contain a carboxyl terminus provided by amino acids 398-431 of SEQ ID NO: 3, indicates that a SEQ ID NO: 1 related region is present and additional polypeptides regions may be present. If additional polypeptide regions are present, then the polypeptide does not have a carboxyl domain provided by amino acids 398-431 of SEQ ID NO: 1.

Percent identity (also referred to as percent identical) to a reference sequence is determined by aligning the polypeptide sequence with the reference sequence and determining the number of identical amino acids in the corresponding regions. This number is divided by the total number of amino acids in the reference sequence (e.g., SEQ ID NO: 1) and then multiplied by 100 and rounded to the nearest whole number.

Another aspect of the present invention describes an immunogen comprising an amino acid sequence that provides protective immunity against S. aureus and one or more additional regions or moieties covalently joined to the polypeptide at the carboxyl terminus or amino terminus, wherein each region or moiety is independently selected from a region or moiety having at least one of the following properties: enhances the immune response, facilitates purification, or facilitates polypeptide stability.

Reference to “additional region or moiety” indicates a region or moiety different from a PBP4 region. The additional region or moiety can be, for example, an additional polypeptide region or a non-peptide region.

Another aspect of the present invention describes a composition able to induce protective immunity against S. aureus in a patient. The composition comprises a pharmaceutically acceptable carrier and an immunologically effective amount of an immunogen that provides protective immunity against S. aureus.

An immunologically effective amount is an amount sufficient to provide protective immunity against S. aureus infection. The amount should be sufficient to significantly prevent the likelihood or severity of a S. aureus infection.

Another aspect of the present invention describes a nucleic acid comprising a recombinant gene encoding a polypeptide that provides protective immunity against S. aureus. A recombinant gene contains recombinant nucleic acid encoding a polypeptide along with regulatory elements for proper transcription and processing (which may include translational and post translational elements). The recombinant gene can exist independent of a host genome or can be part of a host genome.

A recombinant nucleic acid is nucleic acid that by virtue of its sequence and/or form does not occur in nature. Examples of recombinant nucleic acid include purified nucleic acid, two or more nucleic acid regions combined together that provides a different nucleic acid than found in nature, and the absence of one or more nucleic acid regions (e.g., upstream or downstream regions) that are naturally associated with each other.

Another aspect of the present invention describes a recombinant cell. The cell comprises a recombinant gene encoding a polypeptide that provides protective immunity against S. aureus.

Another aspect of the present invention describes a method of making a polypeptide that provides protective immunity against S. aureus. The method involves growing a recombinant cell containing recombinant nucleic acid encoding the polypeptide and purifying the polypeptide.

Another aspect of the present invention describes a polypeptide that provides protective immunity against S. aureus made by a process comprising the steps of growing a recombinant cell containing recombinant nucleic acid encoding the polypeptide in a host and purifying the polypeptide. Different host cells can be employed.

Another aspect of the present invention describes a method of inducing a protective immune response in a patient against S. aureus. The method comprises the step of administering to the patient an immunologically effective amount of an immunogen providing protective immunity against S. aureus.

Unless particular terms are mutually exclusive, reference to “or” indicates either or both possibilities. Occasionally phrases such as “and/or” are used to highlight either or both possibilities.

Reference to open-ended terms such as “comprises” allows for additional elements or steps. Occasionally phrases such as “one or more” are used with or without open-ended terms to highlight the possibility of additional elements or steps.

Unless explicitly stated reference to terms such as “a” or “an” is not limited to one. For example, “a cell” does not exclude “cells”. Occasionally phrases such as one or more are used to highlight the possible presence of a plurality.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 illustrates the amino acid sequence of SEQ ID NO: 2. The portion shown in bold is SEQ ID NO: 1. The underlined regions are added to SEQ ID NO: 1 to provide SEQ ID NO: 2.

FIGS. 2A and 2B illustrate a sequence comparison between SEQ ID NO: 1, SEQ ID NO: 3, SEQ ID NO: 4, SEQ ID NO: 5, SEQ ID NO: 6, SEQ ID NO: 7, and SEQ ID NO: 8. Amino acid differences between the sequences are shown in bold.

FIG. 3 illustrates a nucleic acid sequence (SEQ ID NO: 9) encoding SEQ ID NO: 2. The region encoding SEQ ID NO: 1 is shown in bold. Regions encoding additional sequences are underlined.

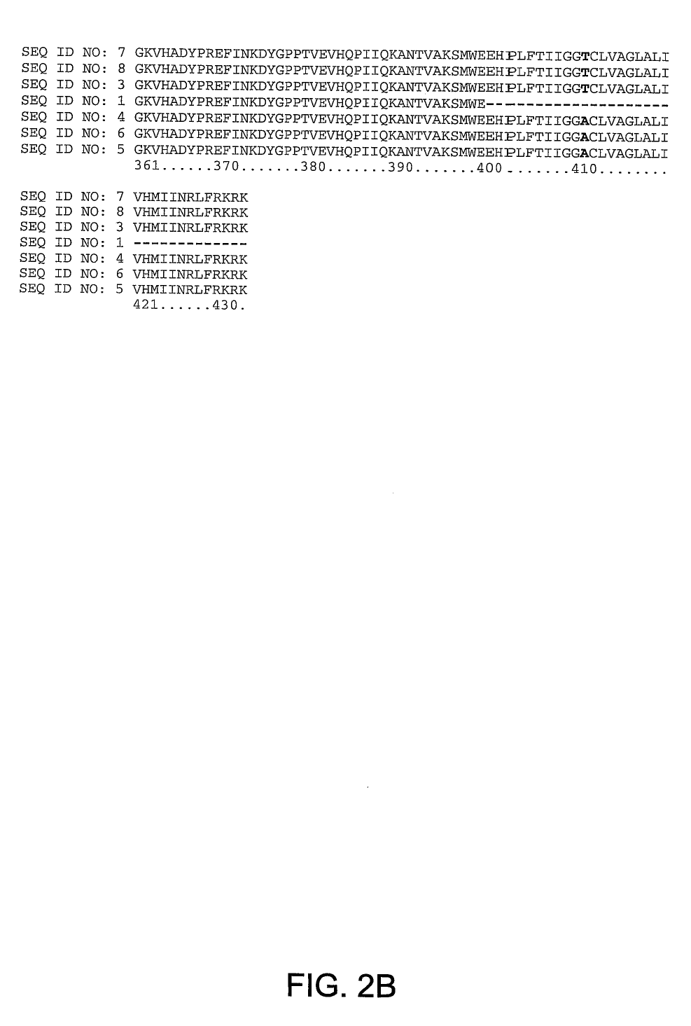

FIG. 4 illustrate survival data using a SEQ ID NO: 2 polypeptide in aluminum hydroxyphosphate adjuvant (ABP).

### DETAILED DESCRIPTION OF THE INVENTION

The ability of SEQ ID NO: 1 related polypeptides to provide protective immunity is illustrated in the Examples provided below using SEQ ID NO: 2. SEQ ID NO: 2 is a derivative of SEQ ID NO: 1 containing an amino terminus methionine-alanine-serine addition and a carboxyl His-Tag. The amino terminus addition resulted from modification to the encoding sequence to provide restriction sites. The His-Tag facilitates polypeptide purification and identification.

SEQ ID NO: 1 is a derivative of PBP4. The full-length PBP4 polypeptide sequence is provided by SEQ ID NO: 3. SEQ ID NO: 1 differs from SEQ ID NO: 3 by lacking the amino terminus methionine and amino acids 398-431. Amino acids 398-431 is a putative carboxyl hydrophobic region involved in membrane binding.

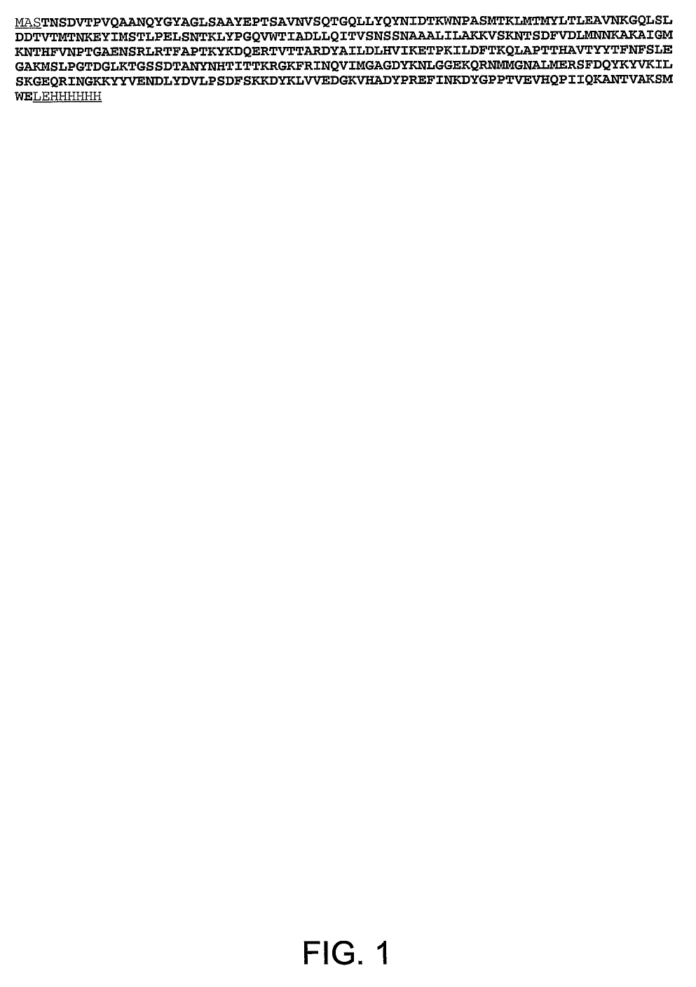

Polypeptides structurally related to SEQ ID NO: 1 include polypeptides containing corresponding regions present in different S. aureus strains and derivatives of naturally occurring regions. The amino acid sequence of SEQ ID NO: 1 is illustrated by the bold region in FIG. 1. FIG. 1 also illustrates the amino terminus methinonine-alanine-serine addition and a carboxyl His-Tag, which are present in SEQ ID NO: 2.

PBP4 Sequences

PBP4 is a pencillin-bind protein. (Henze et al., Antimicrobial Agents and Chemotherapy 39:2415-2422, 1995; Domanski et al., Gene 167:111-113, 1995; Henze et al., Antimicrobial Agents and Chemotherapy 40:2121-2125, 1996; Henze et al., Microbial Drug Resistance 2:193-199, 1996; Domanski et al., Journal of Bacteriology 179:2651-2657, 1997.)

Other naturally occurring PBP4 sequences can be identified based on the presence of a high degree of sequence similarity or contiguous amino acids compared to a known PBP4 sequence. Contiguous amino acids provide characteristic tags. In different embodiments, a naturally occurring PBP4 sequence is a sequence found in a Staphylococcus, preferably S. aureus, having at least 20, at least 30, or at least 50 contiguous amino acids as in SEQ ID NO: 1; and/or having at least 85% sequence similarity or identity with SEQ ID NO: 1.

Sequence similarity can be determined by different algorithms and techniques well known in the art. Generally, sequence similarity is determined by techniques aligning two sequences to obtain maximum amino acid identity, allowing for gaps, additions and substitutions in one of the sequences.

Sequence similarity can be determined, for example, using a local alignment tool utilizing the program lalign (developed by Huang and Miller, Adv. Appl. Math. 12:337-357, 1991, for the <<sim>> program). The options and environment variables are:-f # Penalty for the first residue a gap (-14 by default); -g # Penalty for each additional residue in a gap (-4 by default)-s str (SMATRIX) the filename of an alternative scoring matrix file. For protein sequences, PAM250 is used by default-w # (LILEN) output line length for sequence alignments (60).

SEQ ID NO: 1 Related Polypeptides

A SEQ ID NO: 1 “related” polypeptide contains a region structurally related to a full-length PBP4 or a fragment thereof. SEQ ID NO: 1 related polypeptides are polypeptides having at least about 85% sequence identity to a corresponding region of a naturally occurring PBP4. Reference to “polypeptide” does not provide a minimum or maximum size limitation.

A polypeptide at least 85% identical to SEQ ID NO: 1 contains up to about 56 amino acid alterations from SEQ ID NO: 1. In different embodiments, the SEQ ID NO: 1 related polypeptide is at 90%, at least 94%, or at least 99% identical to SEQ ID NO: 1; differs from SEQ ID NO: 1 by 0, 1, 2, 3, 4, 5, 6, 7, 8, 9, 10, 11, 12, 13, 14, 15, 16, 17, 18, 19, or 20 amino acid alterations; or consists essentially of amino acids SEQ ID NO: 1. Each amino acid alteration is independently an addition, deletion or substitution.

Reference to “consists essentially” of indicated amino acids indicates that the referred to amino acids are present and additional amino acids may be present. The additional amino acids can be at the carboxyl or amino terminus. In different embodiments 1, 2, 3, 4, 5, 6, 7, 8, 9, 10, 11, 12, 13, 14, 15, 16, 17, 18, 19, or 20 additional amino acids are present. In additional embodiments, the polypeptide consists of SEQ ID NO: 1, methionine-SEQ ID NO: 1, alanine-serine-SEQ ID NO: 1 or methionine-alanine-serine-SEQ ID NO: 1.

Alterations can be made to SEQ ID NO: 1 to obtain derivatives that can induce protective immunity against S. aureus. Alterations can be performed, for example, to obtain a derivative retaining the ability to induce protective immunity against S. aureus or to obtain a derivative that in addition to providing protective immunity also has a region that can achieve a particular purpose.

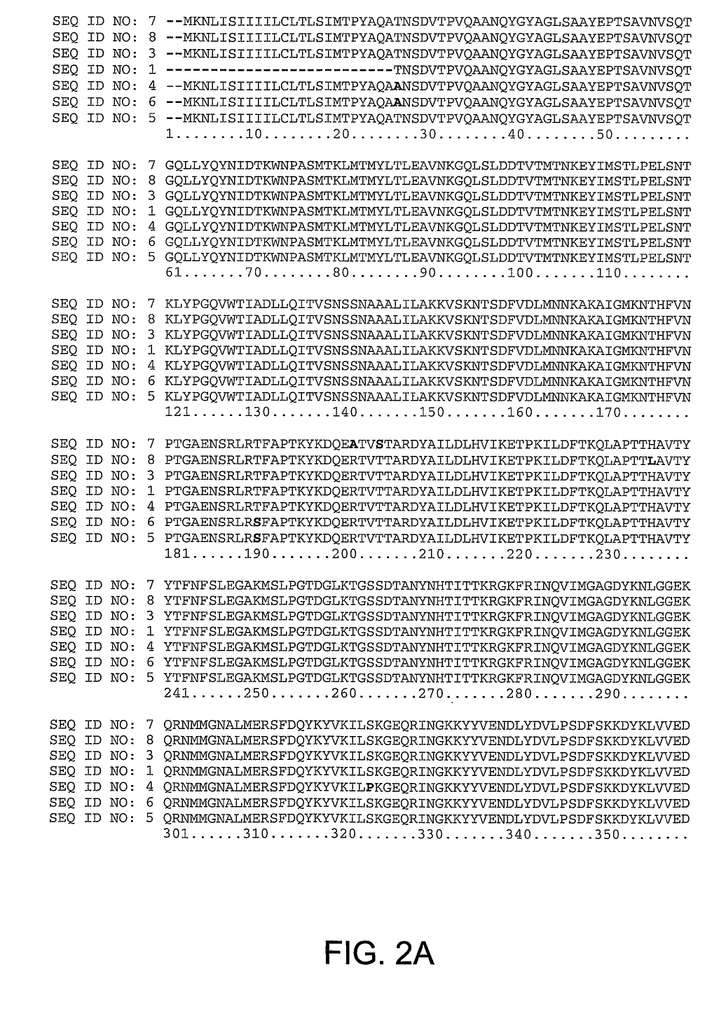

The sequence comparison provided in FIG. 2 can be used to guide the design of potential alterations to SEQ ID NO: 1. FIG. 2 provides different naturally occurring sequences: SEQ ID NO: 3 (COL, SA0699; www.tigr.org); SEQ ID NO: 4 (MSRA.SAR0652, Holden et al., PNAS 101(26):9786-9791, 2004); SEQ ID NO: 5 (SAV0642, Kuroda et al., Lancet 357:1225-1240, 2001); SEQ ID NO: 6 (MW2, MW0604 Baba et al., Lancet 359:1819-1827, 2002, and MSSA, SAS608 Holden et al., PNAS 101(26):9786-9791, 2004); SEQ ID NO: 7 (U29454, Domanski et al. Gene 167:111-113 1995); and SEQ ID NO: 8 (X91786, Henze et al., Antimicrobial Agents and Chemotherapy 40: 2121-2125, 1996).

In addition, alterations can be made taking into account other PBP4 sequences and known properties of amino acids. Generally, in substituting different amino acids to retain activity it is preferable to exchange amino acids having similar properties. Factors that can be taken into account for an amino acid substitution include amino acid size, charge, polarity, and hydrophobicity. The effect of different amino acid R-groups on amino acid properties are well known in the art. (See, for example, Ausubel, Current Protocols in Molecular Biology, John Wiley, 1987-2002, Appendix IC.)

In exchanging amino acids to maintain activity, the replacement amino acid should have one or more similar properties such as approximately the same charge and/or size and/or polarity and/or hydrophobicity. For example, substituting valine for leucine, arginine for lysine, and asparagine for glutamine are good candidates for not causing a change in polypeptide functioning.

Alterations to achieve a particular purpose include those designed to facilitate production or efficacy of the polypeptide; or cloning of the encoded nucleic acid. Polypeptide production can be facilitated through the use of an initiation codon (e.g., coding for methionine) suitable for recombinant expression. The methionine may be later removed during cellular processing. Cloning can be facilitated by, for example, the introduction of restriction sites which can be accompanied by amino acid additions or changes.

Efficacy of a polypeptide to induce an immune response can be enhanced through epitope enhancement. Epitope enhancement can be performed using different techniques such as those involving alteration of anchor residues to improve peptide affinity for MHC molecules and those increasing affinity of the peptide-MHC complex for a T-cell receptor. (Berzofsky et al., Nature Review of immunology 1:209-219, 2001.)

Preferably, the polypeptide is a purified polypeptide. A “purified polypeptide” is present in an environment lacking one or more other polypeptides with which it is naturally associated and/or is represented by at least about 10% of the total protein present. In different embodiments, the purified polypeptide represents at least about 50%, at least about 75%, or at least about 95% of the total protein in a sample or preparation.

In an embodiment, the polypeptide is “substantially purified.” A substantially purified polypeptide is present in an environment lacking all, or most, polypeptides with which the polypeptide is naturally associated. For example, a substantially purified S. aureus polypeptide is present in an environment lacking all, or most, other S. aureus polypeptides. An environment can be, for example, a sample or preparation.

Reference to “purified” or “substantially purified” does not require a polypeptide to undergo any purification and may include, for example, a chemically synthesized polypeptide that has not been purified.

Polypeptide stability can be enhanced by modifying the polypeptide carboxyl or amino terminus. Examples of possible modifications include amino terminus protecting groups such as acetyl, propyl, succinyl, benzyl, benzyloxycarbonyl or t-butyloxycarbonyl; and carboxyl terminus protecting groups such as amide, methylamide, and ethylamide.

In an embodiment of the present invention the polypeptide immunogen is part of an immunogen containing one or more additional regions or moieties covalently joined to the polypeptide at the carboxyl terminus or amino terminus, where each region or moiety is independently selected from a region or moiety having at least one of the following properties: enhances the immune response, facilitates purification, or facilitates polypeptide stability. Polypeptide stability can be enhanced, for example, using groups such as polyethylene glycol that may be present on the amino or carboxyl terminus.

Polypeptide purification can be enhanced by adding a group to the carboxyl or amino terminus to facilitate purification. Examples of groups that can be used to facilitate purification include polypeptides providing affinity tags. Examples of affinity tags include a six-histidine tag, trpE, glutathione and maltose-binding protein.

The ability of a polypeptide to produce an immune response can be enhanced using groups that generally enhance an immune response. Examples of groups that can be joined to a polypeptide to enhance an immune response against the polypeptide include cytokines such as IL-2. (Buchan et al., 2000. Molecular Immnunology 37:545-552.)

Polypeptide Production Polypeptides can be produced using standard techniques including those involving chemical synthesis and those involving purification from a cell producing the polypeptide. Techniques for chemical synthesis of polypeptides are well known in the art. (See e.g., Vincent, Peptide and Protein Drug Delivery, New York, N.Y., Decker, 1990.) Techniques for recombinant polypeptide production and purification are also well known in the art. (See for example, Ausubel, Current Protocols in Molecular Biology, John Wiley, 1987-2002.)

Obtaining polypeptides from a cell is facilitated using recombinant nucleic acid techniques to produce the polypeptide. Recombinant nucleic acid techniques for producing a polypeptide involve introducing, or producing, a recombinant gene encoding the polypeptide in a cell and expressing the polypeptide.

A recombinant gene contains nucleic acid encoding a polypeptide along with regulatory elements for polypeptide expression. The recombinant gene can be present in a cellular genome or can be part of an expression vector.

The regulatory elements that may be present as part of a recombinant gene include those naturally associated with the polypeptide encoding sequence and exogenous regulatory elements not naturally associated with the polypeptide encoding sequence. Exogenous regulatory elements such as an exogenous promoter can be useful for expressing a recombinant gene in a particular host or increasing the level of expression. Generally, the regulatory elements that are present in a recombinant gene include a transcriptional promoter, a ribosome binding site, a terminator, and an optionally present operator. A preferred element for processing in eukaryotic cells is a polyadenylation signal.

Expression of a recombinant gene in a cell is facilitated through the use of an expression vector. Preferably, an expression vector in addition to a recombinant gene also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Due to the degeneracy of the genetic code, a large number of different encoding nucleic acid sequences can be used to code for a particular polypeptide. The degeneracy of the genetic code arises because almost all amino acids are encoded by different combinations of nucleotide triplets or “codons”. Amino acids are encoded by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=ilstidine: codons CAC, CAU
I=Ile=soleucine: codons AUA, AUC, AUU
K=Lys=Lysine: codons AAA, AAG
L=Leu=Leucine: codons UUA, WLUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Suitable cells for recombinant nucleic acid expression of SEQ ID NO: 1 related polypeptides are prokaryotes and eukaryotes. Examples of prokaryotic cells include E. coli; members of the Staphylococcus genus, such as S. aureus; members of the Lactobacillus genus, such as L. plantarum; members of the Lactococcus genus, such as L. lactis; and members of the Bacillus genus, such as B. subtilis. Examples of eukaryotic cells include mammalian cells; insect cells; yeast cells such as members of the Saccharomyces genus (e.g., S. cerevisiae), members of the Pichia genus (e.g., P. pastoris), members of the Hansenula genus (e.g., H. polymorpha), members of the Kluyveroinyces genus (e.g., K. lactis or K. fragilis) and members of the Schizosaccharomyces genus (e.g., S. pombe).

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2^{nd} Edition, Cold Spring Harbor Laboratory Press, 1989.

If desired, expression in a particular host can be enhanced through codon optimization. Codon optimization includes use of more preferred codons. Techniques for codon optimization in different hosts are well known in the art.

SEQ ID NO: 1 related polypeptides may contain post translational modifications, for example, N-linked glycosylation, O-linked glycosylation, or acetylation. Reference to “polypeptide” or an “amino acid” sequence of a polypeptide includes polypeptides containing one or more amino acids having a structure of a post-translational modification from a host cell, such as a mammalian, insect or yeast host cell.

Post translational modifications can be produced chemically or by making use of suitable hosts. For example, in S. cerevisiae the nature of the penultimate amino acid appears to determine whether the N-terminal methionine is removed. Furthermore, the nature of the penultimate amino acid also determines whether the N-terminal amino acid is N^{∝}-acetylated (Huang et al., Biochemistry 26:8242-8246, 1987). Another example includes a polypeptide targeted for secretion due to the presence of a secretory leader (e.g., signal peptide), where the polypeptide is modified by N-linked or O-linked glycosylation. (Kukuruzinska et al., Ann. Rev. Biochem. 56:915-944, 1987.)

Adjuvants

Adjuvants are substances that can assist an immunogen in producing an immune response. Adjuvants can function by different mechanisms such as one or more of the following: increasing the antigen's biologic or immunologic half-life; improving antigen delivery to antigen-presenting cells; improving antigen processing and presentation by antigen-presenting cells; and inducing production of immunomodulatory cytokines. (Vogel, Clinical Infectious Diseases 30 (suppl. 3):S266-270, 2000.)

A variety of different types of adjuvants can be employed to assist in the production of an immune response. Examples of particular adjuvants include aluminum hydroxide, aluminum phosphate, or other salts of aluminum, calcium phosphate, DNA CpG motifs, monophosphoryl lipid A, cholera toxin, E. coli heat-labile toxin, pertussis toxin, muramyl dipeptide, Freund's incomplete adjuvant, MF59, SAF, immunostimulatory complexes, liposomes, biodegradable microspheres, saponins, nonionic block copolymers, muramyl peptide analogues, polyphosphazene, synthetic polynucleotides, IFN-γ, IL-2, IL-12, and ISCOMS. (Vogel Clinical Infectious Diseases 30 (suppl 3):S266-270, 2000, Klein et al., Journal of Pharmaceutical Sciences 89:311-321, 2000, Rimmelzwaan et al., Vaccine 19:1180-1187, 2001, Kersten Vaccine 21:915-920, 2003, O'Hagen Curr. Drug Target Infect. Disord., 1:273-286, 2001.)

Patients for Inducing Protective Immunity

A “patient” refers to a mammal capable of being infected with S. aureus. A patient can be treated prophylactically or therapeutically. Prophylactic treatment provides sufficient protective immunity to reduce the likelihood, or severity, of a S. aureus infection. Therapeutic treatment can be performed to reduce the severity of a S. aureus infection.

Prophylactic treatment can be performed using a vaccine containing an immunogen described herein. Such treatment is preferably performed on a human. Vaccines can be administered to the general population or to those persons at an increased risk of S. aureus infection.

Persons with an increased risk of S. aureus infection include health care workers; hospital patients; patients with a weakened immune system; patients undergoing surgery; patients receiving foreign body implants, such a catheter or a vascular device; patients facing therapy leading to a weakened immunity; and persons in professions having an increased risk of burn or wound injury. (The Staphylococci in Human Disease, Crossley and Archer (ed.), Churchill Livingstone Inc. 1997.) Non-human patients that can be infected with S. aureus include cows, pigs, sheep, goats, rabbits, horses, clogs, cats and mice. Treatment of non-human patients is useful in protecting pets and livestock, and in evaluating the efficacy of a particular treatment.

Combination Vaccines

SEQ ID NO: 1 related polypeptides can be used alone, or in combination with other immunogens, to induce an immune response. Additional immunogens that may be present include: one or more additional S. aureus immunogens, such as those referenced in the Background of the Invention supra; one or more immunogens targeting one or more other Staphylococcus organisms such as S. epidennidis, S. haemolyticus, S. wamneri, or S. lugunensis; and one or more immunogens targeting other infections organisms.

Animal Model System

An animal model system was used to evaluate the efficacy of an immunogen to produce a protective immune response against S. aureus. The animal model was a slow kinetics lethality model involving S. aureus prepared from cells in stationary phase, appropriately titrated, and intravenously administered. This slow kinetics of death provides sufficient time for the specific immune defense to fight off the bacterial infection (e.g., 10 days rather 24 hours).

S. aureus cells in stationary phase can be obtained from cells grown on solid medium. They can also be obtained from liquid, however the results with cells grown on solid media were more reproducible. Cells can conveniently be grown overnight on solid medium. For example, S. aureus can be grown from about 18 to about 24 hours under conditions where the doubling time is about 20-30 minutes.

S. aureus can be isolated from solid or liquid medium using standard techniques to maintain S. aureus potency. Isolated S. aureus can be stored, for example, at −70° C. as a washed high density suspension (>10⁹ colony forming units (CFU)/mL) in phosphate buffered saline containing glycerol.

The S. aureus challenge should have a potency providing about 80 to 90% death in an animal model over a period of about 7 to 10 days starting on the first or second day. Titration experiments can be performed using animal models to monitor the potency of the stored S. aureus inoculum. The titration experiments can be performed about one to two weeks prior to an inoculation experiment.

Administration

Immunogens can be formulated and administered to a patient using the guidance provided herein along with techniques well known in the art. Guidelines for pharmaceutical administration in general are provided in, for example, Vaccines Eds. Plotkin and Orenstein, W. B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990, each of which are hereby incorporated by reference herein.

Pharmaceutically acceptable carriers facilitate storage and administration of an immunogen to a patient. Pharmaceutically acceptable carriers may contain different components such as a buffer, sterile water for injection, normal saline or phosphate buffered saline, sucrose, histidine, salts and polysorbate.

Immunogens can be administered by different routes such as subcutaneous, intramuscular, or mucosal Subcutaneous and intramuscular administration can be performed using, for example, needles or jet-injectors.

Suitable dosing regimens are preferably determined taking into account factors well known in the art including age, weight, sex and medical condition of the patient; the route of administration; the desired effect; and the particular compound employed. The immunogen can be used in multi-dose vaccine formats. It is expected that a dose would consist of the range of 1.0 μg to 1.0 mg total polypeptide, in different embodiments of the present invention the range is 0.01 mg to 1.0 mg and 0.1 mg to 1.0 mg.

The timing of doses depends upon factors well known in the art. After the initial administration one or more booster doses may subsequently be administered to maintain or boost antibody titers. An example of a dosing regime would be day 1, 1 month, a third dose at either 4, 6 or 12 months, and additional booster doses at distant times as needed.

Generation of Antibodies

A SEQ ID NO: 1 related polypeptide can be used to generate antibodies and antibody fragments that bind to the polypeptide or to S. aureus. Such antibodies and antibody fragments have different uses including use in polypeptide purification, S. aureus identification, or in therapeutic or prophylactic treatment against S. aureus infection.

Antibodies can be polyclonal or monoclonal. Techniques for producing and using antibodies are well known in the art. Examples of such techniques are described in Ausubel, Current Protocols in Molecular Biology, John Wiley, 1987-2002, Harlow et al., Antibodies, A Laboratory Manual, Cold Spring Harbor Laboratory, 1988, and Kohler et al., Nature 256:495-497, 1975.

### EXAMPLES

Examples are provided below further illustrating different features of the present invention. The examples also illustrate useful methodology for practicing the invention. These examples do not limit the claimed invention.

### Example 1

### Protective Immunity

This example illustrates the ability of SEQ ID NO: 1 related polypeptides to provide protective immunity in an animal model. SEQ ID NO: 2, a derivative of SEQ ID NO: 1, was used to provide protective immunity.

SEQ ID NO: 2 Cloning and Expression

The protein was designed to be expressed from the pET24b vector (Novagen) with the His tag residues encoded by the vector. The vector provides the initiating Met codon followed by Ala and Ser codons at the N-terminus and adds Leu, Glu, (His)6 and a termination codon at the C-terminus (amino acids 378-385 of SEQ ID NO: 2). The designed DNA sequence encodes a 385 amino acid altered form of mature pbp4 (COL-SA0699).

The pbp4 DNA sequence was translated using Vector NTI software and the resulting amino acid sequence (SEQ ID NO: 1) was analyzed. PCR primers were designed to amplify the gene starting at the 25^{th} residue (Thr) to remove a signal sequence and to end at the 398^{th} codon (Glu) to remove the C-terminal membrane binding site. The forward PCR primers contained an NheI restriction site to facilitate cloning into the expression vector. The reverse PCR primer included a XhoI restriction site to facilitate cloning into the expression vector.

PCR amplified sequences were digested with NheI and XhoI then ligated into the pET24b vector (Novagen) digested with the same two enzymes. The ligation reaction was transformed into E. coli strain NovaBlue and colonies selected by growth in LB with 50 μg/mL kanamycin. DNA minipreps were made (Qiagen), and insert integrity determined by restriction digestion and sequence confirmation. A clone was selected containing no DNA changes from the desired sequence.

E. coli BLR(DE3) cells (Novagen) were transformed and grown on LB plates containing kanamycin (50 μg/ml). Liquid LB (kanamycin) cultures were set up by inoculating with single colonies from the LB (kanamycin) plates and incubated at 37° C., 250 rpm overnight. The overnight culture was diluted back into fresh LB (kanamycin), grown until the A₆₀₀ was between 0.6 and 1.0, an un-induced cell pellet harvested and the rest of the culture induced by the addition of IPTG to final concentrations of 1 mM and allowed to express for three hours. Cell pellets were harvested from 1 ml culture by centrifugation at 5000×g for 3 minutes at room temperature. Cell pellets were resuspended in 200 μl lysis buffer (Bugbuster, with protease inhibitors, Novagen), then soluble and insoluble fractions of the induced samples were obtained. An equal volume of loading buffer (supplemented with β-mecapto ethanol to 5% final volume) was added prior to heating the samples at 90° C. for 3 minutes. Extracts were run on Novex 4-12% Tris-Glycine gels and assayed for protein (Coomassie Blue stained) and blotted onto nitrocellulose and probed with anti-HIS6 antibodies (Zymed).

SEQ ID NO: 2 Purification

Frozen recombinant E. coli cell paste (39.7 grams) was thawed and resuspended in 100 ml Lysis Buffer (Protease inhibitor cocktail (Roche #1873580, 1 tablet per 10 grams of cell paste), 50 mM sodium phosphate, pH 8.0, 0.15 M NaCl, 2 mM magnesium chloride, 10 mM imidazole, 0.1% Tween-80, and 0.02% sodium azide.

Benzonase (250 Units/microliter, EM #1.10697.0002) was added to the cell suspension at 1 microliter per gram of cell paste. A lysate was prepared with a microfluidizer. The lysate was stirred at 4° C. for 2.5 hours. The lysate was clarified by centrifugation at 10,000×g for 10 minutes at 4° C. The supernatant was filtered through a 5-micron glass-fiber filter (Millipore #AP2504700). NaCl was added to the filtrate to a final concentration of 0.5 M from a 5 M stock solution. The filtrate was mixed with Ni-NTA agarose chromatography resin (Qiagen #30250) and stirred overnight at 4° C.

The slurry of chromatography resin was poured into a chromatography column and the non-bound fraction was collected by gravity from the column outlet. The column was washed with 50 mM sodium phosphate, pH 8.0, 0.5 M NaCl, 2 mM magnesium chloride, 10 mM imidazole, and 0.1% Tween-80, and the column was eluted with Elution Buffer (0.3 M imidazole, 0.15 M NaCl, and 20 mM Tris-HCl, pH 8.0). Fractions containing protein product were identified by dot blotting on nitrocellulose membrane with Ponceau-S staining. Protein-containing fractions were pooled to make the Ni-IMAC Product. The Ni-IMAC Product was fractionated by SEC and product-containing fractions were identified by SDS/PAGE with Coomassie staining. SEC fractions of highest purity and concentration were pooled to make the SEC Product. The SEC Product was sterile-filtered and adsorbed on aluminum hydroxyphosphate adjuvant at a final concentration of 0.2 mg/ml.

Preparation of S. aureus Challenge

S. aureus was grown on TSA plates at 37° C. overnight. The bacteria were washed from the TSA plates by adding 5 ml of PBS onto a plate and gently resuspending the bacteria with a sterile spreader. The bacterial suspension was spun at 6000 rpm for 20 minutes using a Sorvall RC-5B centrifuge (DuPont Instruments). The pellet was resuspended in 16% glycerol and aliquots were stored frozen at −70° C.

Prior to use, inocula were thawed, appropriately diluted and used for infection. Each stock was titrated at least 3 times to determine the appropriate dose inducing slow kinetics of death in naive mice. The potency of the bacterial inoculum (80 to 90% lethality) was constantly monitored to assure reproducibility of the model. Ten days before each challenge experiment, a group of 10 control animals (immunized with adjuvant alone) were challenged and monitored.

Protection Studies for a SEQ ID NO: 2 Polypeptide

Twenty BALB/c mice were immunized with three dose& of a SEQ ID NO: 2 polypeptide (20 μg per dose) on aluminum hydroxyphosphate adjuvant (450 μg per dose). Aluminum hydroxyphosphate adjuvant (AHP) is described by Klein et al., Journal of Pharmaceutical Sciences 89, 311-321, 2000. The doses were administered as 100 microliters containing 20 micrograms intramuscular injections (50 microliters per leg) on days 0, 7 and 21. The mice were bled on day 28, and the antibody serum responses to SEQ ID NO: 2 were quantified by ELISA.

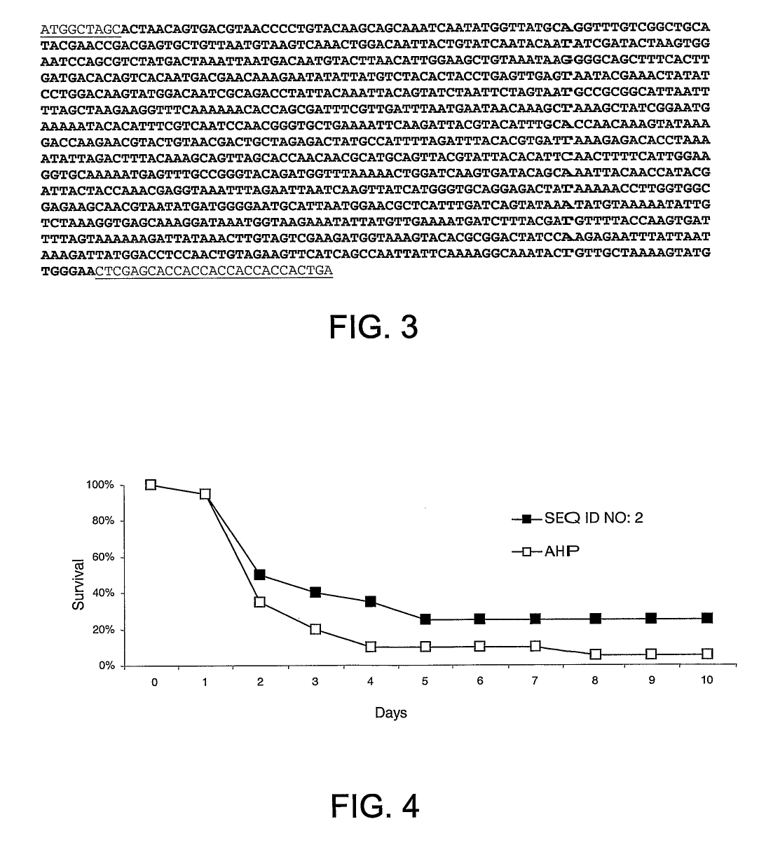

On day 35 of the experiment the mice were challenged with S. aureus (5-9×10⁸ CFU/mouse) and evaluated against a control set of 20 mice that had just been immunized with AIIP. The mice were monitored over a 10 day period for survival. At the end of the experiment 5 mice survived the SEQ ID NO: 2 immunized group compared to 1 surviving in the ABP control group. The results are illustrated in FIG. 4.

Other embodiments are within the following claims. While several embodiments have been shown and described, various modifications may be made without departing from the spirit and scope of the present invention.

1. A polypeptide immunogen comprising an amino acid sequence at least 85% identical to SEQ ID NO: 1, wherein said polypeptide provides protective immunity against S. aureus and wherein if one or more additional polypeptide regions are present said additional regions do not provide a carboxyl terminus containing amino acids 398-431 of SEQ ID NO: 3.

2. The polypeptide of claim 1, wherein said polypeptide consists of an amino acid sequence at least 94% identical to SEQ ID NO: 1.

3. The polypeptide of claim 2, wherein said polypeptide consists essentially of amino acids 4-385 of SEQ ID NO: 1.

4. The polypeptide of claim 3, wherein said polypeptide consists of an amino acid sequence of SEQ ID NO: 1, methionine-SEQ ID NO: 1, alanine-serine-SEQ ID NO: 1 or methionine-alanine-serine-SEQ ID NO: 1.

5. An immunogen comprising an amino acid sequence at least 85% identical to SEQ ID NO: 1 and one or more additional regions or moieties covalently joined to said amino acid sequence at the carboxyl terminus or amino terminus, wherein each region or moiety is independently selected from a region or moiety having at least one of the following properties: enhances the immune response, facilitates purification, or facilitates polypeptide stability.

6. A composition able to induce a protective immune response in a patient comprising an immunologically effective amount of the immunogen of claim 1 and a pharmaceutically acceptable carrier.

7. The composition of claim 6, wherein said composition further comprises an adjuvant.

8. A nucleic acid comprising a recombinant gene comprising a nucleotide sequence encoding the polypeptide of claim 1.

9. The nucleic acid of claim 8, wherein said nucleic acid is an expression vector.

10. A recombinant cell comprising a recombinant gene comprising a nucleotide sequence encoding the polypeptide of claim 1.

11. A method of making a S. aureus polypeptide that provides protective immunity comprising the steps of:
(a) growing the recombinant cell of claim 10 under conditions wherein a polypeptide is expressed; and
(b) purifying said polypeptide.

12. A method of inducing a protective immune response in a patient comprising the step of administering to said patient an immunologically effective amount of immunogen comprising an amino acid sequence at least 85% identical to SEQ ID NO: 1.

13. The method of claim 12, wherein said patient is a human.

14. The method of claim 13, wherein said patient is treated prophylactically against S. aureus infection.

15. A method of inducing a protective immune response in a patient comprising the step of administering to said patient an immunologically effective amount of a polypeptide made by the method of claim 11.
